(19) United States
(12) Patent Application Publication
(10) Pub. No.: US 2010/0041906 A1
(43) Pub. Date: Feb. 18, 2010

(54) METHOD FOR THE PRECIPITATION OF ORGANIC COMPOUNDS

(75) Inventors: Huibert Albertus Van Boxtel, Tilburg (NL); Servatius Hubertus Johannes Wilhelmus Leenen, Valkenswaard (NL); Matheus Lambertus Spapens, Veldhoven (NL)

Correspondence Address: FOLEY AND LARDNER LLP;SUITE 500, 3000 K STREET NW, WASHINGTON, DC, 20007, US

(73) Assignees: FUJI-FILM MANUFACTURING EUROPE B.V.; FUJI PHOTO FILM B.V.

(21) Appl. No.: 12/162,278

(22) PCT Filed: Jan. 26, 2007
(86) PCT No.: PCT/NL07/00028

§ 371 Date: Dec. 5, 2008

(30) Foreign Application Priority Data
Jan. 26, 2006 (EP) 06100907.2
Feb. 14, 2006 (EP) 06101651.5

(51) Int. Cl. C07J 5/00; C07C 231/22; C07J 75/00; C07J 3/00; C07J 1/00

(52) U.S. Cl. 552/595; 564/223; 552/611; 552/623

(57) ABSTRACT

The present invention concerns a method for the controlled precipitation of organic compounds giving crystals with a very small average size and a very narrow size distribution.

### FIELD OF THE INVENTION

The present invention is in the field of precipitation of substances. It relates generally to the technical field of methods for the controlled nucleation and growth of crystals, in particular crystallisation of organic substances.

### BACKGROUND OF THE INVENTION

Crystallization from solution is an important separation and purification process in chemical process industries. It is the primary method for the production of a wide variety of materials ranging from inorganic compounds, such as calcium carbonate and soda ash, to high added value materials, such as pharmaceuticals and specialty chemicals.

In the pharmaceutical industry, crystallization from solution of pharmaceutically active compounds or their intermediates is the typical method of purification. It is in this industry very important to obtain the desired crystal average size, size distribution, morphology, polymorph and purity of the active ingredient. In the case of drugs that are slightly soluble in water the crystal size strongly affects the dissolution rate and equilibrium solubility in water. These factors reflect the drug bioavailability in the human body.

Crystallization from solution begins with the nucleation of crystals followed by the growth of these nuclei to finite size. Nucleation and growth follow separate kinetic regimes with nucleation normally occurring at high driving forces (over-saturation) and growth occurring at all levels of over-saturations. The growth rate is usually faster at increasing over-saturation levels. Beyond a critical over-saturation there will be spontaneous nucleation of new nuclei. The direct crystallization of small sized high surface area particles is usually accomplished in a high saturation environment which often results in material of low purity, high friability, and decreased stability due to poor crystal structure formation. Because the bonding forces in organic crystal lattices generate a much higher frequency of amorphism than those found in highly ionic inorganic solids, “oiling out” of over-saturated material is not uncommon, and such oils often solidify without structure.

Slow crystallization is a common technique used to increase product purity and produce a more stable crystal structure, but it is a process that decreases crystallizer productivity and produces large, low surface area particles that require subsequent high intensity milling. Currently, pharmaceutical compounds almost always require a post-crystallization milling step to increase particle surface area and thereby improve their bioavailability. However, high energy milling has drawbacks. Milling may result in excessive local temperatures resulting in degradation of material, yield loss, noise and dusting, as well as unwanted personnel exposure to highly potent pharmaceutical compounds. Also, stresses generated on crystal surfaces during milling can adversely affect labile compounds. Overall, the three most desirable end-product goals: 1) high surface area, 2) high chemical purity and 3) high stability are notoriously difficult to optimize simultaneously using current crystallization technology without high energy milling.

In order to get the smallest possible crystals one needs to maximize the over-saturation and to find out the critical over-saturation value. The critical over-saturation needs to be determined for each precipitating compound and each precipitating condition (such as type of solvent, temperature, etc.). A major problem with the usual crystallization methods of organic compounds is that it can be difficult to obtain high over-saturations. High over-saturation (S), wherein S is defined as the actual concentration of a substance divided by the concentration when the substance in the particular solvent at a certain temperature is just saturated, means a value of S higher than for example 1.5.

Another problem can be the extremely rapid nucleation rate, being faster than the mixing time. Estimated nucleation rates of milli-, or microseconds or even nano seconds are not uncommon for solvent/anti-solvent and for reaction precipitations. Nucleation rates can be estimated using classical nucleation theory, see Kashchiev (D. Kashchiev, Nucleation, Basic Theory with Applications, Butterworth-Heinemann, 2000) and Kashchiev and van Rosmalen (D. Kashchiev and G. M. van Rosmalen, Review: Nucleation in solutions revisited, Cryst. Res. Technol., 38, No. 7-8, 555-574, 2003), taking into account improved estimates for the particle-solution interfacial energy, see Granberg et al (R. A. Granberg, C. Ducreux, S. Gracin and A. C. Rasmuson, Primary nucleation of paracetamol in acetone-water mixtures, Chem. Eng. Sci., 56, 2305-2313, 2001). In case of solvent anti-solvent precipitation over-saturations can become extremely high especially when applying the so-called “reverse” addition sequence. Reverse addition means adding the solution of the organic compound to the anti-solvent. This typically creates a higher over-saturation than adding the anti-solvent to the solution of the organic compound. An over-saturation S of 10 or more is considered extremely high in this context.

In the field of photography methods and apparatus are known for the preparation of silver halide crystals. A particular method for the preparation these crystals makes use of a mixing chamber into which an aqueous solution of a halide and an aqueous solution of a silver salt are separately and simultaneously added. The mixing chamber is positioned in a larger growth or chamber into which the silver halide nuclei are discharged to grow further into the desired silver halide crystals. Suitable apparatus for carrying out such a method for producing silver halide crystals are described in U.S. Pat. No. 4,289,733, EP 523842, EP 708362, EP 1357423, EP 0 709 723, US 2003/0224308, U.S. Pat. No. 6,050,720 and U.S. Pat. No. 5,202,226. The methods and apparatus cited above always deal with the problem of obtaining silver halide crystals, having a specific structure and with a narrow crystal size distribution.

In the field of crystallisation of organic, pharmaceutically active compounds, U.S. Pat. No. 5,314,506 uses an impinging jet mixer to generate small crystal sizes with the majority of crystals in the size range of 3 to 20 microns (μm). This method requires the use of surfactants to inhibit agglomeration of individual particles. Agglomeration increases the effective particle size and thus lowers the bioavailability of the product.

EP 1 157 726 uses the impinging jet device with reaction precipitation. The use of a sonication probe to enhance micro mixing in the fluid contacting area is suggested but no examples are given for its positive effect.

U.S. Pat. No. 6,302,958 uses a sonication probe in the immediate vicinity of impinging jets to generate small crystal sizes of less than 1 micron. However, in this application the use of surfactants is advocated to alleviate agglomeration of particles during the precipitation process. Furthermore the solvent anti-solvent system claimed is DMSO and water respectively. DMSO however is not preferred as solvent in pharmaceutical compound precipitations due to its toxicity.

Because the methods, used nowadays for the crystallization of organic compounds in general, and pharmaceutical compounds more in particular, have many disadvantages, there remains a need for a method providing the creation of organic particles with a small average size and a reproducible and narrow size distribution without the aid of surfactants or polymers or the use of toxic chemicals.

### SUMMARY OF THE INVENTION

In their search for efficient and reproducible methods for the crystallisation of organic, pharmaceutically active compounds into very small crystals with a narrow crystal size distribution, the present inventors came to the surprising insight that an optimal result can be achieved using a mixing chamber placed and in open connection with a larger vessel comprising a non solvent to which mixing chamber a solution of the organic compound is added and the mixing chamber is provided with stirring means, which provides for isotropic turbulent mixing in said mixing chamber. Further it is essential that in the mixing chamber an over-saturated solution of the substance that is to be precipitated is formed or introduced in order to allow the formation of precipitated nuclei of the organic compounds.

It was found, that especially conditions should be provided of high over-saturation in the mixing chamber together with a residence time of the organic compound in said mixing chamber which is at least longer than the induction time, by which nucleation and growth substantially only occurs in the mixing chamber and not outside the mixing chamber in the vessel. Methods to measure induction times are described in Kashchiev and van Rosmalen, Review: Nucleation in solutions revisited, Cryst. Res. Technol., 38, No. 7-8, 2003.

The vessel comprises in essence the non solvent and the mixing chamber is placed below the non solvent liquid level of the vessel. In order to have sufficient mixing in the mixing chamber and still a residence time long enough in order to let the nucleation occur in the mixing chamber, the inventors found out, that mixing means providing for vigorous isotropic mixing can advantageously be used. The preferred mixing means are characterised by low axial flows at very high rotation speed by which the mixing in the mixing chamber is highly efficient and the residence time of the organic compounds in the mixing chamber can be adjusted.

Thus in particular the invention concerns a method for the controlled precipitation of an organic compound comprising the steps of providing a solution of said organic compound, adding said solution via one or more inlets into a mixing chamber, which is provided with one or more stirring means each comprising one or more stirrer blades and a shaft, capable of providing isotropic mixing, said mixing chamber being positioned inside and in open connection with a vessel, which vessel comprises a liquid in which the organic compound will not dissolve, or in other words is a non-solvent, and the mixing chamber is positioned below the surface level of said non-solvent, wherein the addition of said solution to the mixing chamber provides for over-saturation of said organic compound in the mixture in said mixing chamber resulting in crystallisation and growth of crystals of said organic compound and wherein the stirring means is operative and provides for isotropic mixing and provides for a residence time of said organic compound in said mixing chamber which is longer than 0.1 millisecond.

The main advantage of the method of the invention is that a very small crystal size is obtained having a narrow size distribution by which further milling is not required anymore. Another advantage of this technology is that crystals can be produced with a smaller average size and narrower size distribution than is the case with common crystallisation techniques. Thus the result of the present invention is that crystals with small average sizes and narrow size distributions can be produced.

Although in the present method surfactants and/or polymers can be present during the nucleation and/or growth step, an advantage of the present invention is, that there is no need for the use of these compounds during crystallisation in order to prevent agglomeration. The presence of surfactants during the crystal nucleation stage is disadvantageous in case the initial particle size generated is too small and the particles need to be grown to somewhat bigger size in a second step. Surface adsorbing additives like surfactants or polymers can inhibit growth of the crystal faces. This is not preferred when the desired size is larger than the initial size obtained during the precipitation step. With the current invention surfactant and/or polymer can be added just after nucleation and growth are finished. The purpose of surfactant and/or polymer is to keep the suspension from sedimentation and flocculation.

Another advantage of the present invention, is that the organic compound crystallises very purely, without inclusion of impurities.

The phrase “a liquid in which the organic compound will not dissolve, or in other words is a non-solvent” should not be interpreted absolutely. The skilled person will realise that solubility depends on certain conditions such as for example temperature. Said phrase refers to precipitation of the organic compound of interest under which the process is normally carried out. Preferably the precipitation is to such an extent that an economically viable yield of the compound of interest can be obtained.

### DETAILED DESCRIPTION OF THE INVENTION

The term ‘organic compounds’ in its broadest sense refers to compounds containing carbon atoms. Usually an organic compound also contains hydrogen atoms. Very often organic compounds also contain oxygen and/or nitrogen atoms and to a lesser extent sulphur atoms. In particular the term ‘organic compounds’ refers what is normally considered an organic compound in the field of pharmaceutical, dye, agricultural and chemical industry. This includes ‘biological’ organic compounds such as hormones proteins and the like. Herein below organic compound(s) is also referred to as substance(s).

The term ‘precipitation’ refers to a subclass of the field of solution crystallisation. Precipitation is recognised by one or more of the following characteristics: (i) low solubility of the crystallizing compound, (ii) fast process, (iii) small crystal size and (iv) irreversibility of the process (W. Gerhartz in: Ullmans encyclopedia of Industrial Chemistry, vol. B2 5^{th} ed., VHC Verlagsgessellschaft mbH, Weinheim, FGR, 1988). In the context of this invention a suitable definition for precipitation is the relatively rapid formation of a sparingly soluble solid phase from a liquid solution phase (Handbook of Industrial crystallization, Edited by Allan S. Myerson, Butterworth Heinemann, Oxford, p 141).

Generally two types of processes resulting in precipitation can be discerned:
A first type of process is anti-solvent (also referred to as non-solvent) precipitation. A dissolved substance is mixed with a solvent that lowers its solubility so that a precipitate will form. A modification of the anti-solvent precipitation is that a dissolved substance is not necessarily mixed with an anti-solvent but is mixed in such way that the solubility of the precipitating solvent is lowered such that nuclei are formed. This can be realised by variations in for example temperature, pH (addition of acid or alkaline solutions), ionic strength and the like and combinations of such factors.
A second type of process is reaction precipitation. Two components are mixed resulting in the formation of a newly formed substance and due to the low solubility of the formed substance under the used mixing or reaction conditions a precipitate will form.

With the term ‘over-saturation’ is meant a concentration of a substance that is in excess of saturation under the given conditions, i.e. solvent or solvent mixture, temperature, pH, ionic strength etc.

A solution of the substance(s) to be precipitated is inserted into a mixing chamber. The mixing chamber is provided with agitation means, in particular stirring means, providing an axial flow and a radial flow. Preferably the stirring means can be controlled. Preferably mixing chamber and/or vessel are provided with temperature control means. The mixing chamber is positioned inside, and in open connection with a vessel. The position of the mixing chamber can be anywhere in the vessel as long as there is an open connection, outlet, of the mixing chamber with the vessel. Preferably the mixing chamber is below the solvent surface in the vessel. Also preferably the mixing chamber is in the lateral middle of the vessel where the vertical position can be varied from bottom till just below the solvent surface. Prior to the precipitation, the same solvent is present in the mixing chamber and in the vessel. Via one or more inlets the substance to be precipitated, or components that form a substance to be precipitated, preferably dissolved in a solvent or solvent mixture is introduced into the mixing chamber resulting together with the axial flow provided by the stirring means in a net outflow from the mixing chamber into the vessel. Two, three, four or even more inlets (nozzles) may be present.

In one embodiment all parts of the nucleation apparatus that are in contact with the over-saturated solutions, or with the bulk solution containing the crystals, are coated with a layer of a material that prevents adhering, fouling, incrustation and such. For example, the inner wall of the vessel and all parts of the agitator and mixing chambers in contact with the solution are coated with for example polytetrafluoroethylene (PTFE), in particular Teflon®, and the like. In general coating material having a low surface tension can be advantageously used. This is not a strict requirement, as in the present invention, by selecting the proper conditions, fouling or encrustation is of minor importance.

In one embodiment of the method of the invention a solution of the substance to be precipitated is introduced into the mixing chamber through one or more inlets. In a further embodiment at the same time separately a non-solvent for the substance to be precipitated is introduced into the mixing chamber.

The precipitate can thus also be formed by the simultaneous addition of a solution of the substance to be precipitated and a non-solvent in the mixing chamber. In one embodiment the solution that is present in the vessel and mixing chamber at the start of the precipitation process is a mixture of the used solvent and non-solvent or a mixture of non solvents. In a specific embodiment the solution that is present in the vessel and mixing chamber at the start of the precipitation process is saturated with the substance to be precipitated. The ratio of solvent to non-solvent which is used depends on the solvent and non solvent used, the substance to be crystallised and the crystal size one likes to obtain. An important factor is the amount of over-saturation. Over-saturation in this respect is defined as the actual concentration divided by the equilibrium concentration, meaning the concentration where the solution is just saturated. Depending on the compound to be crystallised over-saturation levels of more than 1.5, even more than 2.5 and for some compounds as high as 10 and even more can be advantageous. For some substances even over-saturation levels of 100 are more can be used. The over-saturation can be controlled by the stirring speed, residence time, temperature, concentration of the organic compound in the solution and the like.

In yet another embodiment of the method of the invention the substance to be precipitated is formed in the mixing chamber in a chemical reaction. In a particular embodiment the substance to be precipitated is formed by reaction from two or more components. More specific the substance to be precipitated is formed by a substantially instantaneous chemical reaction involving the formation of covalent and/or ionic bonds such as protonation/deprotonation, anion/cation exchange, acid addition salt formation/liberation, or any other type of chemical reaction. In this embodiment, the liquid in the vessel is also a non solvent for the compound which is formed.

The volumes of the mixing chamber and the vessel may vary from smaller than 10 milliliters, to several liters to more than 1000 liters. A suitable chamber/vessel ratio of volumes may vary for instance from less than 0.001 to 0.1.

The size of the mixing chamber depends very much on the scale at which one wants to perform the crystallisation. On small scale (1-5 dm³ vessel) one typically would use a mixing chamber of 10-150 cm³, for medium scale (5-500 dm³ vessel) a mixing chamber of 150-500 cm³ and so on as long as the above mentioned chamber vessel ratio is maintained. The shape of the mixing chamber can be chosen freely and in case it is rotational symmetric around a central axis can for example be specified by two identical surfaces one top surface and one bottom surface, at a distance x from each other which surfaces may have any shape from rectangular to dodecahedral or even cylindrical with, when applicable, a minimum diameter of Dmin. For example, for a mixing chamber having a square shape, Dmin is the distance between opposite sides). In this embodiment, x can be larger than Dmin and alternatively, x can also be smaller than Dmin. In a further embodiment, the top surface and bottom surface need not to be identical, but one surface can be for example of a smaller size than the other.

At the start of the nucleation, the nuclei are surrounded by over-saturated fluid. When two or more of these particles stay in contact for too long, they will be “cemented” together to an agglomerate. Furthermore, unlike inorganic particles in aqueous media, organic particles most often are not electrically charged and therefore these organic particles do not have a repulsive mechanism. In the present invention the drag/shear forces in the mixing chamber imposed on the nuclei by the turbulent fluid motion prevents the particles from agglomerating. It is an aim of this invention to use excessive turbulence to reduce the inter-particle contact times to values that do not allow agglomeration while the surrounding fluid is still over-saturated. The mixing can be characterized by the Reynolds number N_{Re}, which is given by the equation:

$${N}_{Re} = \frac{{Da}^{2} \cdot N \cdot ρ}{μ}$$

wherein
Da=the blade diameter (m);
N=rotational speed (r/s);
ρ=fluid density (g/cm³); and
μ=viscosity (Pa*s).

Typically the flow in a stirred tank is laminar when N_{Re} is smaller than 10. There is a transition region between laminar and turbulent flow when 10<N_{Re}<10⁴ and the flow is isotropically turbulent when N_{Re} is larger than 10⁴, see Perry (Perry's Chemical Engineers' Handbook, Ed.: R. H. Perry and D. W. Green, McGraw-Hill, Ch 18, 1999).

Without being bound by theory it is assumed, that upon maximizing N_{Re}, even beyond the point of isotropic turbulence, the shear stresses exerted on particles will increase, causing a de-agglomerating effect. A second advantage of the isotropic high turbulence is, that it will result in the reactants to get mixed faster, by which the over-saturation will reduce to a level, where no nucleation occurs anymore in the mixture which leaves the mixing chamber. It is assumed that this results in a narrower size distribution of crystals with a smaller average size than when the mixing of fluids is incomplete in the mixing chamber. It has been observed for example, that sphere like crystal agglomerations occur at insufficient mixing, suggesting nucleation at the surface of the solution droplets entering the mixing chamber from the inlet and which are dissipated too slow in the non-solvent by the insufficient mixing

From the formula we can conclude, that the Reynolds number increases at higher stirrer blade diameter. In the present invention it was found, that a preferred size of stirrer blade is at least 50% and more preferably at least 70% and most preferably between 80 and 95% of the smallest dimension of the mixing chamber. Very good results were obtained with a stirrer blade which had a diameter of around 90% of the smallest dimension of the mixing chamber. In case of a cylindrical shaped mixing chamber the dimension refers to the diameter of the round top or bottom surface. In case of for example a cubical shaped mixing chamber the dimension refers to length of the side of a square top or bottom surface or the dimension refers to the length of the shortest side of a rectangular top or bottom surface.

In this invention, stirrer blades having a Reynolds number of at least 10⁴ should be used, preferably more than 10⁵ and even more preferably more than 10⁶. The current invention creates extreme turbulence in the region of first contact of the solution of the organic compound and anti-solvent to guarantee efficient and fast micro mixing by which in the mixing chamber in principal a homogeneous mixture is available. Furthermore, the forces acting on freshly generated nuclei due to this turbulence are such, that contact times between particles are short enough to limit agglomeration of these particles.

Surprisingly it was found, that stirring with very high rotation speeds in a small mixing chamber provides for conditions at which very small crystals are obtained with a narrow particle (crystal) size distribution. Without being bound to theory it is assumed, that nucleation and growth (also named induction) are essentially molecular level processes, so only mixing on the molecular scale can directly influence the process. The micro-mixing time, this is the time at which in general a homogeneous mixture is obtained in the mixing chamber is described for example by Bal/dyga et al, Chem. Eng. Sci., vol. 50, No. 8, pp 1281-1300, 1995.

The authors describe a micro mixing time (τ_{ω}) with the formula

$${τ}_{ω} = 12 \cdot \sqrt{\frac{v}{ɛ}} ,$$

in which ν=kinematic viscosity and ε=the rate of energy dissipation per unit mass.

This equation suggests that maximizing the power input by the agitation device, e.g. stirring means, in a smallest possible mixing chamber, combined with a low viscosity of the solvent/anti-solvent mixture, minimizes the micro-mixing time.

In case of very fast nucleation, the micro-mixing time should be very low. If in the latter case the mixing time is too long unwanted agglomeration of crystals might occur. The current invention further aims at keeping the mixed reactants in the mixing chamber at least long enough to allow nucleation and growth to a level at which the crystals have grown to a stable size and the over-saturation of the solution surrounding the crystals is low enough to stop nucleation.

The selected stirring means, or stirrer, should rotate at very high speed in order to reach the required Reynolds number. The stirrer speed should be at least above 1,000 rpm (rotations per minute) more preferably above 5,000 rpm and even more preferably above 10,000 rpm giving isotropic turbulent mixing. Also rotation speeds as high as 15,000 rpm can advantageously be used.

To those skilled in the art, it will be surprising, that these high speeds can be used in a small mixing chamber. In general the shape of the stirrer blade can be chosen freely, taking into account however that the shape of stirrer blades determines amongst others the ratio between axial flow (perpendicular to the stirrer blade) and radial (parallel to the stirrer blade) flow. With only axial flow, no mixing will occur in the mixing chamber, while with only radial flow there will be no or limited outflow of the mixing chamber. The mixing blades can be made of any material which does not give deformation at high stirring speeds and high shear forces. Preferably the material is stainless steel which might or might not be coated with a surface energy lowering coating, like Teflon®. Multiple blades can be mounted on the same stirrer axis or on separate stirrer axes, rotating in the same direction or counter rotating. In another embodiment two impellers are mounted in the mixing chamber. Both impellers can comprise one or more stirrer blades on the same or a different axis, while the blades of the impellers can be on the same height or have a distance towards each other within about 50% of the total height of the mixing chamber.

A suitable geometry of the stirrer blade can be selected by the following test:

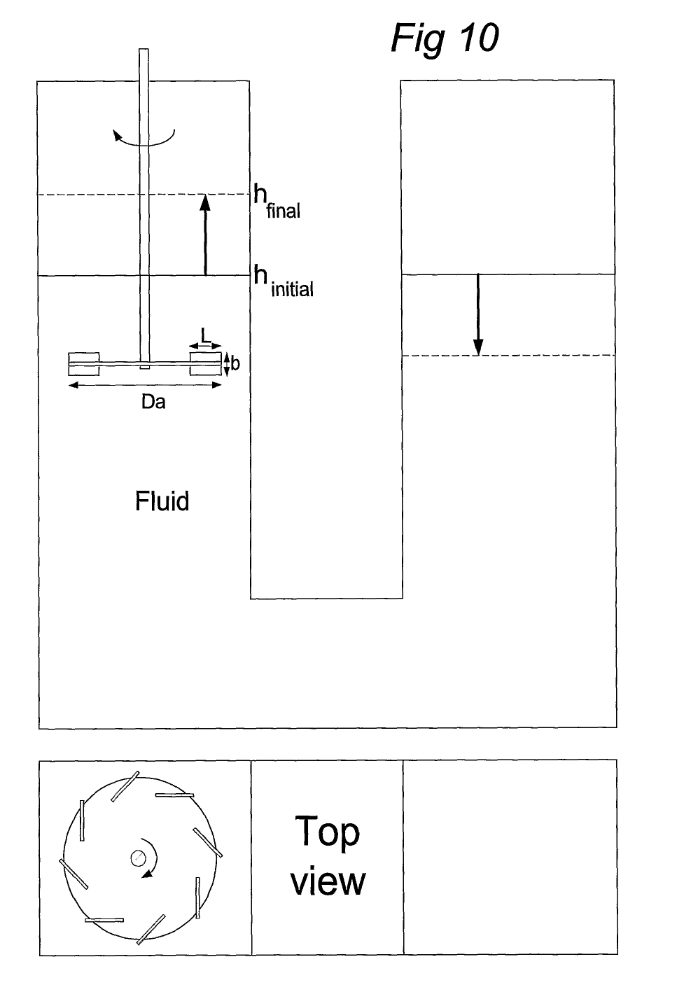

An impeller (meaning a shaft and a stirrer blade) attached to a motor is mounted in a u-shaped transparent vessel (see FIG. 10). Upon rotating the impeller the fluid height in both vertical tube sections will change, in opposite directions. In case the fluid temperature within the whole device is held constant, the well known Toricelli's law (eq 1.) is used to relate the average fluid velocity, discharged axially by the impeller, to the fluid level change.

ν̅²̅=2·g·Δh (Eq. 1)

in which ν̅²̅ is the average squared axially discharged fluid velocity, g is the gravitational constant and Δh is the total fluid height difference between both vertical tube sections.

$$Via {N}_{q} = \frac{\overline{v} \cdot A}{N \cdot {Da}^{3}} \qquad ( Eq . 2 )$$

in which A is the tube cross-sectional area, the axial discharge flow Q (l/min.) of the impeller can be deduced by

Q=N_{q}·N·Da³ (Eq. 3)

In case of a plain disk as a stirrer blade, the axial flow will be 0. In case of propeller like stirrer blades, the axial flow will be too high, by which the residence time in the mixing chamber will become too low.

The chamber residence time tᵣₑₛ can be approximated by:

$${t}_{res} = \frac{V}{Q} \qquad ( Eq . 4 )$$

in which V is the chamber volume.

With the condition that tᵣₑₛ should be at least 0.1 milliseconds, the skilled person can determine what is a suitable stirrer blade.

From the above, we can see, that the residence time of the organic compound in the mixing chamber can be varied amongst others by the choice of the type, e.g. shape and size, of the stirring blade and intensity of mixing. A too short residence time in the mixing chamber will result in uncontrolled nucleation outside the mixing chamber due to the feeding of highly over-saturated solution into the vessel. A too long residence time in the mixing chamber can result in excessive agglomeration and growth. In a further embodiment, the residence time is influenced by (partly) blocking the top and/or bottom surfaces of the mixing chamber.

The very high turbulence created in the mixing chamber has the extra advantage that it causes a de-agglomerating effect on the possibly created fine crystal agglomerates. Agglomeration is typically a very strong function of crystal density and over-saturation. In the present invention a very high over-saturation is created in the mixing chamber to generate numerous and fine crystals. Agglomeration of these crystals is to be expected under these conditions when using a normal crystallization vessel and normal stirring speeds. However using the preferred conditions of this invention agglomeration can be prevented and there is no need for taking additional measures to avoid agglomerations such as using a protective colloid. By the proper selection of the shape of the stirring means, the revolutions per second and the size of the mixing chamber, the estimated residence time in the mixing chamber can be varied from microseconds to seconds. In many cases solvent and non-solvent, together with the temperature can be chosen such that the nucleation is very fast, e.g. faster than 1 microsecond and even below 10⁻⁹ seconds. The isotropic turbulent mixing is therefore a very important factor, as with reduced mixing efficiencies at these very high nucleation speeds, agglomeration is almost inevitable.

Also for compounds not having such a fast nucleation time, the residence times in the mixing chamber should not be too long, because the efficiency of the crystallisation process will be low and by the long residence time a wide particle size distribution will be obtained and on average larger crystal sizes. In practice the mixing chamber residence time preferably does not exceed 3 seconds and in most of the cases preferably are below 1 second.

In our experience the residence time in the mixing chamber preferably is at least 0.1 millisecond as with lower residence times growth might be insufficient giving instable particles which tend combine and agglomerate.

In case nucleation proceeds slowly e.g. from 10⁻³ until 10⁻⁶ seconds, the conditions preferably are chosen such that the residence time is more than 10⁻¹ but below for example 5 seconds and more preferably below 3 seconds.

During nucleation it is important that there is an over-saturation which is sufficient to initiate nucleation in the mixing chamber. After the addition of the solution of the organic compound into the mixing chamber comprising the non-solvent has started, the critical over-saturation level at which nucleation starts is obtained within fractions of a second. During the whole crystallisation process this over-saturation should be maintained. By the isotropic turbulent mixing the compound to be crystallised is in a very short time isotropically distributed over the mixing chamber. By this nucleation and growth almost exclusively will occur in the mixing chamber and the concentration in the outflow liquid is reduced to values, where no nucleation occurs anymore.

The position in height of the stirring means in the mixing chamber can be varied. The low end of the chamber, the middle part or the upper part of the chamber are positions at which the stirring means can be effective. The preferred position is as close as possible, preferably at the same height as the inlets via which the solution of the organic compounds and or non solvents are added into the mixing chamber. The preferred positions of the inlets is at the position where the distance to the stirrer blade is lowest. This means for a mixing chamber having a square shaped bottom, that the inlet tubes should be positioned at the sides, preferably in the middle of the sides and not in the corners. In case the inlets are at the same height as the stirrer blade and the inlets are as close as possible to the stirrer blade, a rotation speed of 1000 rpm can be used, but more preferably the rotation speed is above 10,000 rpm. The Reynolds number in this case should be above 10⁴, preferably above 10⁵ and even more preferably above 10⁶. Although the preferred position of the inlet tubes is at the same height as the stirrer blade, these positions can also be different, while still obtaining good results. However in the later case, the position difference between the inlet tubes and the stirrer blade preferably does not differ more than 30% of the total height of the mixing chamber, as with a greater difference it is very difficult to obtain the preferred small crystals. In case the inlet position is different from the position of the stirrer blade, the stirring speed is preferably increased to values of 15,000 rpm or more and Reynolds numbers of 10⁶ or more. The phrase “same height” as used herein above allows for some deviation as the skilled person will appreciate. It means for instance that the centre of the one or more inlet tubes is within a height difference of less then 10%, or 8%, or 6%, or 5%, or 4%, or 3%, or 2% or 1% of the height of the mixing chamber with the center of the height of the stirrer blade.

Thus in particular the present invention concerns a method for the controlled precipitation of an organic compound comprising the steps of providing a solution of said organic compound, adding said solution via one or more inlets into a mixing chamber, which is provided with one or more stirring means each comprising one or more stirrer blades and a shaft, capable of providing isotropic mixing, said mixing chamber being positioned inside and in open connection with a vessel, which vessel comprises a liquid in which the organic compound will not dissolve (non solvent) and the mixing chamber is positioned below the surface level of said non-solvent, wherein the addition of said solution to the mixing chamber provides for an over-saturation S₁₀ of said organic compound in the mixture in said mixing chamber of more than 1.5 resulting in crystallisation and growth of crystals of said organic compound and wherein the stirring means is operative and provides for isotropic mixing characterized by a Reynolds number of at least 10⁶ if the stirrer blade and one or more inlets are not on the same height in the mixing chamber, their height difference being not more than 30% of the height of the mixing chamber or the stirring means provides for isotropic mixing characterized by a Reynolds number of the stirrer blade of at least 10⁴ if the stirrer blade and one or more inlets are positioned at the same height in the mixing chamber and provides for a residence time of said organic compound in said mixing chamber which is longer than 0.1 milliseconds.

The fluid feed velocities and the inlet diameters are not critical. The flow of the solutions which are added to the mixing chamber can be chosen freely, however best results are obtained with rather high flows. In view of the sizes of the precipitation vessel that are possible, and a mixing chamber that can have various sizes, the preferred flow can best be expressed related to the size of the mixing chamber. The preferred injected quantity per second is at least 1% of the volume of the mixing chamber and more preferably at least 5% of the volume of the mixing chamber. (When the mixing chamber has a volume of 100 cm³, a suitable flow can be for example 5 ml/s=300 ml/min). Satisfactory results however are also obtained at reduced flows, as long as the mixing in the mixing chamber is isotropic. The tube inlets can have various diameters. The diameter preferably is below about 10% of the height of the mixing chamber.

Solvent and anti-solvents can be chosen with the only restriction of mutual miscibility in mind. Also mixture of solvents in combination with one non-solvents, or a mixture of non solvents in combination with one solvent, or a mixture of solvents in combination with a mixture of non solvents can be used. However for environmental reasons it is preferred to make the system as simple as possible using one solvent and one anti-solvent. The mixture should of course be chosen such that the solubility of the organic compound in the mixture is significantly lower than in the pure solvent.

It was found that high levels of over-saturation are advantageous in the crystallisation method according to the invention. Because the over saturation as such is difficult to define and because of practical reasons we used the over-saturation ratio S at 10 seconds after the start of addition, defined as:

$${S}_{10} = \frac{{C}_{10}}{{C}_{10 , e}} ,$$

in which C₁₀ equals the calculated concentration of solute at 10 seconds after start of addition, C_{10,e} equals the equilibrium solute concentration of solute at 10 seconds after start of addition. S₁₀ is preferably higher than 1.5. In one embodiment S₁₀ is higher than 2.5 and in another embodiment S₁₀ is higher than 5. Depending on the organic compound to crystallize also higher values can be obtained of S₁₀, for example 10 or higher or 100 or higher or any value in between and even values of more than 100 can be obtained. The method of the present invention can be used for all compounds for which it is possible to have an over-saturation in the mixing chamber, by which nucleation and growth (induction) occurs in the mixing chamber and for which no growth or nucleation any more occurs in the bulk liquid of the vessel.

The reactants mixture is expelled from the mixing chamber into the vessel after a short (usually less than 1 second) but optimal residence time in the mixing chamber. In the vessel, there is no oversaturation and therefore no nucleation or growth occurs in the vessel. The vessel might be provided with anchor impellers in order to enhance bulk mixing and keep suspensions dispersed if necessary.

Furthermore if desired, baffles can be added at any position in the vessel to inhibit air entrainment due to the vortex that can be created by the rotating stirrer axis and blade.

A further method to prevent a vortex is to apply a anti vortex ring (circular plate) on the in the mixing chamber centrally placed impeller. The distance of this anti vortex ring to the top of the mixing chamber can be very small, for example below 1 cm or even below 0.5 cm. Placing such a ring will also influence the residence time and the mixing efficiency.

Using the method of the present invention it is possible, for a certain organic compound to make various sizes of crystals by choosing at a specific solvent anti solvent system different addition flows of the dissolved compound into the mixing chamber, different sizes of the mixing chamber and or different stirring speeds and or stirrer blades (obtaining high Reynolds numbers).

Generally and unexpectedly keeping the amount of over-saturation, stirring speed and other conditions the same, a smaller mixing chamber volume will result in a smaller crystal size. This is of particular interest in cases where even smaller sizes of crystals cannot be obtained using the actual size of mixing chamber and mixing means. Consequently by choosing a larger mixing chamber, larger crystals will be obtained. Thus in a further embodiment the invention relates to a method as described above for the control of the size of a substance to be precipitated by choosing various sizes of mixing chambers.

In one embodiment of this invention, the temperature of the vessel and more in particular the temperature in the mixing chamber preferably is controlled in such a manner that temperature fluctuations will not be more that plus or minus 2° C. from a predetermined set temperature, as the temperature determines, amongst others, the solubility of the organic compound. For example a too large temperature rise of the mixture in the mixing chamber due to dissipated agitation power might cause agglomeration due to subsequent cooling in the vessel. In another embodiment of this invention, the temperature in the mixing vessel is kept lower that the temperature of the mixture in the vessel.

The temperature difference may be 10 degrees Celcius or more than 10 degrees, for example 20 degrees or 30 degrees or even 40 degrees or 50 degrees or even as high as 60 degrees Celsius or more. The temperature of the solution of the substance to be precipitated, or components that form a substance to be precipitated, is mostly higher than that in the mixing chamber/vessel. Owing to the open contact of the mixing chamber with the rest of the vessel it is difficult to apply a temperature difference between the mixing chamber and the rest of the vessel. However, when conditions are chosen well, one is able to generate a lower temperature in the mixing chamber compared to the vessel. When adding a significant amount of a very cold non-solvent and a warm solution of the substance to be precipitated in its solvent into the starting solution in the mixing chamber at ambient temperature, it is possible to achieve a temperature in the mixing chamber that is lower than the temperature outside said mixing chamber during the time of this addition. The lower temperature in the mixing chamber will lower the solubility of the substance to be precipitated and thus increase the over-saturation in the mixing chamber even more than if the temperatures of all solutions would be identical.

Depending on the type, average size, size distribution and yield of the desired crystals the skilled person can select the conditions such as temperature, pH, (anti-)solvent(s), ionic strength, addition flow of the of the substance(s) to be precipitated, concentration of the substance(s) to be precipitated, agitation speed, agitation direction, size of the mixing chamber etc., under which an appropriate over-saturation is established in the mixing chamber. For example conditions that favor high over-saturation are reverse addition, low temperature, high addition flow, high concentration of the organic compound small size mixing chamber. For example conditions that favour low over-saturation are normal addition, low addition flow, low concentration of the organic compound, high temperature, large size mixing chamber and the like.

In general the crystals that are formed in the mixing chamber will have the desired average size and size distribution as a result of the conditions under which the crystallisation occurs. The crystals formed are discharged into the vessel from which they can be harvested once a suitable amount is formed.

Although the method of the present invention results in crystals with a very small average size and a very narrow size distribution, which can not be reached by the conventional methods, circumstances can arise in which the size of the precipitated crystals needs to increase, keeping the size distribution narrow. In such a case the inventive crystallisation method may be followed by a growth stage. Usually it suffices to add the substances(s) to be crystallised at slower rates so that re-nucleation is prevented. A suitable measure to influence the growth stage is by varying the temperature of the content of the vessel. Another means of growing the crystals to larger sizes without re-nucleation is by means of adding very small particles into the vessel. These very fine particles should be much smaller in size than the original precipitated crystals. The very fine particles have a larger solubility than the larger originally present particles. The smaller particles will dissolve and create a relatively mild over-saturation which will cause the original particles to grow without re-nucleation in the mixing chamber or vessel.

Referring to the two types of processes resulting in precipitation described above an example of a precipitation process of the anti-solvent type is as follows: a dissolved substance to be crystallized is injected into the mixing chamber. In the mixing chamber and vessel a non-solvent is present, therefore, a precipitate will form. It is also possible that a mixture of both solvents is present (solvent and non-solvent) in mixing chamber and vessel. During crystallisation the solvent with the crystallizing substance and non-solvent are added simultaneously. Optionally, when crystallizing organic or biochemical compounds for example with the solvent precipitation method, the bulk volume that is present before starting the crystallisation is a mixture of solvent and non-solvent. When using a polar solute in an apolar non-solvent and with inefficient mixing, encrustation might happen. In the method of the present invention the occurrence of encrustation is unlikely due to the applied turbulent isotropic mixing. Even in case in the present method encrustation would occur, parts in the process being in contact with the crystallisation liquid might be coated with a surface energy lowering coating like Teflon®, PVDF and the like

In another embodiment the non-solvent is the same solvent as used to dissolve the crystallizing compound only at another pH, temperature etc. An example of this is the precipitation reaction of sodium L-glutamate. This dissolves well in water at pH 7, however, if this solution is injected in the mixing chamber in combination with injection of an aqueous acidic solution, with an aqueous starting solution to make the resulting pH=3.22, a precipitate of L-glutamic acid will form (at pH=3.22 it is sparingly soluble). This type of precipitation can occur via one inlet (solution of sodium L-glutamic acid injected into a solution to make pH=3.22) or via two inlets (solutions of sodium L-Glutamate+acid solution added simultaneously).

Reaction precipitation is illustrated in a simple form as follows: two (or more) soluble compounds, for example A(aq) and B(aq), are introduced simultaneously and separately into the mixing chamber. Owing to the low solubility of the reaction product of A and B a precipitate will be formed. Reaction: A(aq)+B(aq)→AB(s).

Harvesting of the formed crystals from the vessel occurs according to methods known per se in the art and may include decantation, one or more washing steps, filtration, centrifugation, drying and combinations of these steps.

Analytical techniques for studying and characterizing the crystals include X-ray crystallography, Raman spectroscopy, infrared spectroscopy, solid state nuclear magnetic resonance (SSNMR), scanning electron microscopy, atomic force microscopy (AFM), scanning tunnelling microscopy (STM) and/or density measurements.

Average particle size and particle size distribution can be measured with population analysis of Scanning Electron Microscope photographs and Laser Diffraction measurement techniques.

The method of this invention provides for crystals with a very small size and a very narrow size distribution and can be used to obtain crystals of compounds which are used in medical applications as active pharmaceutical ingredient. This is especially beneficial for medicines, where such crystalline active pharmaceutical ingredient is dispersed in a liquid composition used in pulmonal/transdermal/parenteral and oral applications. Also the present crystals may be advantageous in slow release formulations.

In one embodiment the present method is for the precipitation of a hormone, in particular a steroid hormone. In a further embodiment the present method is for the precipitation of a compound selected from the group consisting of Betamethasone, Betamethasone acetate, Betamethasone disodium phosphate, Chloroprednisone acetate, Corticosterone, Cortisone, Desoxycorticosterone, Desoxycorticosterone acetate, Desoxycorticosterone pivalate, Dexamethasone, Dichlorisone acetate, Fluocinolone acetonide, Fluorohydrocortisone, Fluorometholone, Fluprednisolone, Flurandrenolone, Hydrocortisone, Hydrocortisone acetate, Hydrocortisone sodium succinate, Methylprednisolone, Methylprednisolone sodium succinate, Paramethasone, Paramethasone acetate, Prednisolone, Prednisolone Phosphate sodium, Prednisolone pivalate, Prednisone, Triamcinolone, Triamcinolone acetonide, Triamcinolone diacetate, Androsterone, Fluoxymesterone, Methandrostenolone, Methylandrostenediol, Methyl testosterone, Norethandrolone, Oxandrolone, Oxymetholone, Prometholone, Testosterone, Testosterone cypionate, Testosterone enanthate, Testosterone phenylacetate, Testosterone propionate, Equilenin, Equilin, Estradiol, Estradiol benzoate, Estradiol cypionate, Estradiol dipropionate, Estriol, Estrone, Estrone benzoate, Ethynyl estradiol, Mestranol, Acetoxypregnenolone, Anagestone acetate, Chlormadinone acetate, Dimethisterone, Ethisterone, Ethynodiol diacetate, Flurogestone acetate, Hydroxymethylprogesterone, Hydroxymethylprogesterone acetate, Hydroxyprogesterone, Hydroxyprogesterone acetate, Hydroxyprogesterone caproate, Melengestrol acetate, Norethindrone, Norethindrone acetate, Norethisterone, Norethynodrel, Normethisterone, Pregnenolone, Progesterone, Aldosterone, Hydroxydione sodium, Spironolactone.

### EXAMPLES

In the examples a vessel of 4 litres was used with a mixing chamber of 144 cm³.

### Comparative Example 1

Crystallization of paracetamol from ethanol and n-heptane;

using submerged feed into a square shaped mixing chamber with normal agitation.

The vessel was filled with 900 ml 15% (vol.) ethanol in n-heptane. The temperature was controlled at 25° C. 45.5 Grams of paracetamol dissolved in 350 ml ethanol was added at a feed rate of 25 ml/min simultaneously with the addition of n-heptane at a feed rate of 100 ml/min in the mixing chamber. Both solutions were controlled at 25° C. At the start of addition the mixing chamber and agitating device were completely immersed in the fluid present in the vessel. The mixing chamber and stirring means (agitating device) are described in U.S. Pat. No. 4,289,733. The inlet position of both reactants was at opposite sides of the mixing chamber, below the agitating device stirring at 350 rpm. The impeller Reynolds number N_{Re} was 1.6*10⁵. Simultaneous feeding of both reactant fluids is continued for 14 minutes, after which the precipitate was filtrated, washed with n-heptane and collected to yield 26.7 g solid paracetamol (55% yield). The induction time for crystals to be visually observed is 280 seconds after the addition started. S₁₀=0.12.

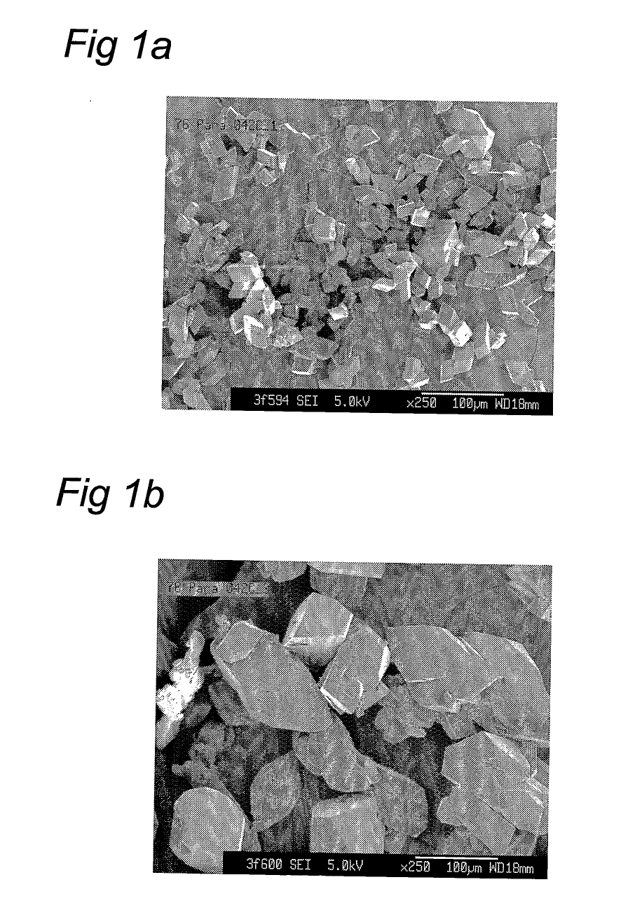

Large crystals were obtained (see FIG. 1) with this low value of S.

### Comparative Example 2

Crystallization of paracetamol from ethanol and n-heptane;

using submerged feed into a square shaped mixing chamber with normal agitation.

The vessel was filled with 900 ml n-heptane. The temperature was controlled at 25° C. 45.5 Grams of paracetamol dissolved in 350 ml ethanol was added at a feed rate of 25 ml/min simultaneously with the addition of n-heptane at a feed rate of 100 ml/min. Both solutions were controlled at 25° C. At the start of addition the mixing chamber and agitating device were completely immersed in the fluid present in the vessel. The mixing chamber and stirring means (agitating device) are described in U.S. Pat. No. 4,289,733.

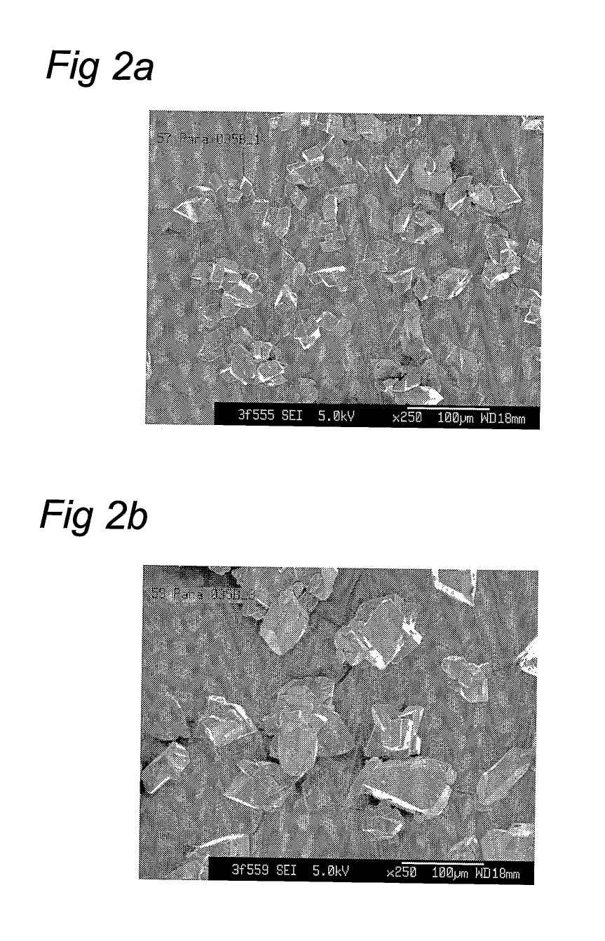

The inlet position of both reactants was at opposite sides of the mixing chamber, below the agitating device stirring at 350 rpm. The impeller Reynolds number N_{Re} was 1.6*10⁵. Simultaneous feeding of both reactant fluids was continued for 13 minutes, after which the precipitate was filtrated, washed with n-heptane and collected to yield 25.2 g suspended solid and 5.5 g of vessel surface “caked” solid paracetamol (in total 74% yield). The induction time for crystals to be visually observed was 20 seconds after the addition started. S₁₀=5.4. As the stirring is insufficient, encrustation occurs and large crystals were obtained (see FIG. 2).

### Comparative Example 3

Crystallization of paracetamol from ethanol and n-heptane,

using submerged feed into a square shaped mixing chamber with high S₁₀ but normal agitation.

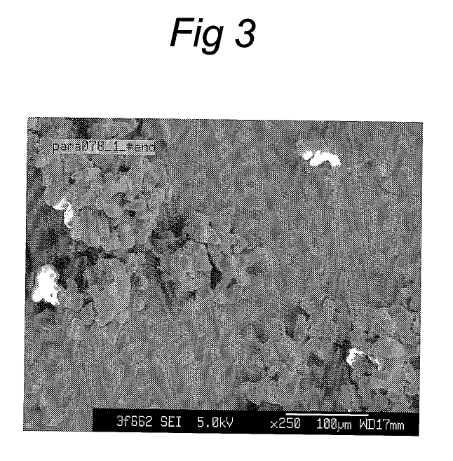

The vessel was filled with 900 ml n-heptane. The initial temperature was controlled at minus 15° C. 45.5 Grams of paracetamol dissolved in 350 ml ethanol was added at a feed rate of 25 ml/min simultaneously with the addition of n-heptane at a feed rate of 100 ml/min. Both solutions were controlled at 25° C. At the start of addition the mixing chamber and agitating device were completely immersed in the fluid present in the vessel. The mixing chamber, vessel and stirring means (agitating device) were PTFE-coated versions of the devices described in U.S. Pat. No. 4,289,733. The inlet position of both reactants was at opposite sides of the mixing chamber, below the agitating device stirring at 350 rpm. The impeller Reynolds number N_{Re} was 1.6*10⁵. Simultaneous feeding of both reactant fluids was continued for 14 minutes, after which the precipitate was filtrated, washed with n-heptane and collected to yield 37.1 g suspended solid without the presence of “caked” solid paracetamol (89% yield). The induction time for crystals to be visually observed was less than 14 seconds after the addition started. S₁₀=13.7. Although no encrustation was observed FIG. 3 shows that mainly unwanted crystal agglomerates were formed.

### Comparative Example 4

Crystallization of paracetamol from ethanol and n-heptane (normal addition order);

using submerged feed into a square shaped mixing chamber with inventive agitation means.

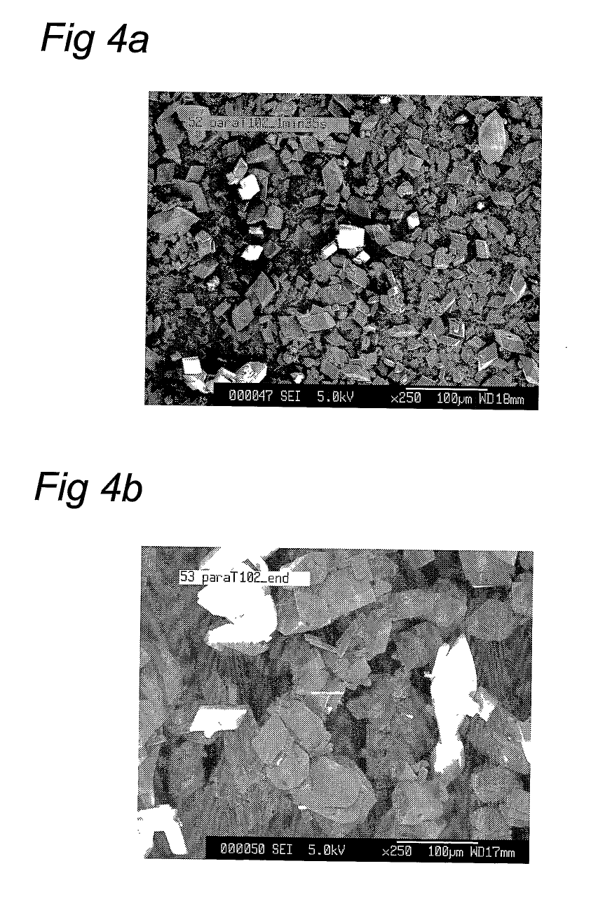

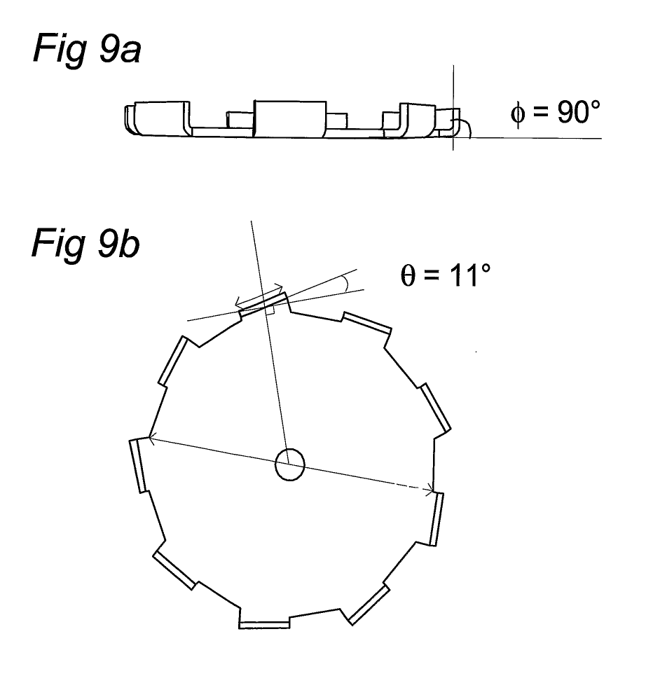

The vessel was filled with a solution of 162.5 g of paracetamol in 1250 ml ethanol. The temperature was controlled at 25° C. 2500 mL n-heptane was added at a feed rate of 1000 ml/min equally divided over two inlets, controlled at 25° C. At the start of addition the mixing chamber and agitating device were completely immersed in the fluid present in the vessel. The mixing chamber and vessel are described in U.S. Pat. No. 4,289,733. In this example as stirring means the stirring blade was used as shown in FIG. 9. Stirring was done at 14,000 rpm, creating tremendous turbulence in the mixing chamber. The impeller Reynolds number N_{Re} was 2.2*10⁶. Simultaneous feeding through both inlets was continued for 2.5 minutes, after which the precipitate was filtrated, washed with n-heptane and collected to yield 81 g suspended solid (in total 50% yield). The induction time for crystals to be visually observed was 80 seconds after the addition started. S₁₀=0.91. FIG. 4 shows, that also in this embodiment large crystals were formed, probably because the required over-saturation could not be reached.

### Comparative Example 5

Crystallization of paracetamol from ethanol and n-heptane, with medium agitation speed (reverse addition order);

using submerged feed into a square shaped mixing chamber with inventive agitation means.

The vessel was filled with 2333 ml n-heptane. The initial temperature was controlled at 25° C. 151 Grams of paracetamol dissolved in 1167 ml ethanol was added at a feed rate of 1000 ml/min equally divided over two inlets, controlled at 25° C. At the start of addition the mixing chamber and agitating device were completely immersed in the fluid present in the vessel. The mixing chamber and vessel are described in U.S. Pat. No. 4,289,733. In this example as stirring means the stirring blade was as depicted in FIG. 9. Stirring was done at 3,000 rpm, creating little turbulence in the mixing chamber. The impeller Reynolds number N_{Re} was 9.3*10⁵. The inlet position of both reactants was at opposite sides of the mixing chamber, 7 mm lower than the height of the stirrer blade. This off-set in inlet height versus impeller height causes an ineffective mixing as can be seen in the SEM photos of the batch. Simultaneous feeding of both reactant fluids was continued for 70 seconds, after which the precipitate was filtrated, washed with n-heptane and collected to yield 84.9 g suspended solid with average size much larger than 10 μm. The induction time for crystals to be visually observed was less than 2 seconds after the addition started. S₁₀=4.5.

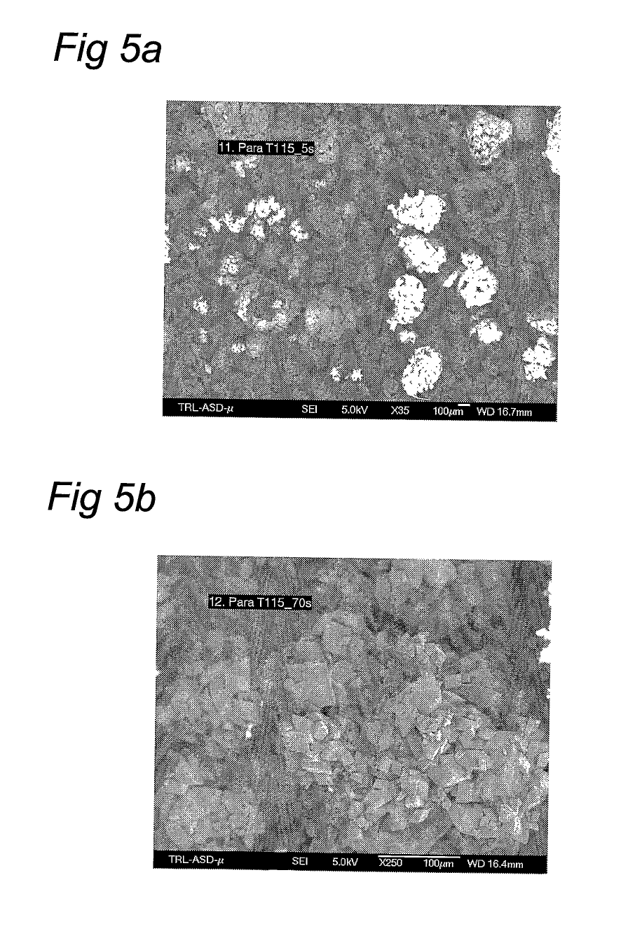

From FIG. 5 it could be concluded that the crystallization speed (nucleation time) under these conditions was clearly faster than the mixing rate, causing hollow spherical crystal structures. Apparently, at the interface of the paracetamol/ethanol solution droplets and the solution in the mixing chamber fast nucleation and growth caused crystals to appear before the droplets were dispersed and mixed with the environment. The final crystal size was significantly larger than in inventive example 1. Better results could be obtained with the same Reynolds number, putting stirrer blade and inlet at the same height or by increasing the Reynolds number by using a bigger stirring blade or by increasing the rotation speed.

### Inventive Example 1

Crystallization of paracetamol from ethanol and n-heptane (reverse addition order);

using submerged feed into a square shaped mixing chamber with inventive agitation means.

The vessel was filled with 2333 ml n-heptane. The initial temperature was controlled at 25° C. 151 Grams of paracetamol dissolved in 1167 ml ethanol was added at a feed rate of 1000 ml/min equally divided over two inlets, controlled at 25° C. At the start of addition the mixing chamber and agitating device were completely immersed in the fluid present in the vessel. The mixing chamber and vessel are described in U.S. Pat. No. 4,289,733. In this example the stirring blade was as depicted in FIG. 9. Stirring was done at 14,000 rpm, creating tremendous turbulence in the mixing chamber. The impeller Reynolds number N_{Re} was 6.6*10⁶. The inlet position of both reactants was at opposite sides of the mixing chamber, at identical height as the stirrer blade. Simultaneous feeding of both reactant fluids was continued for 70 seconds, after which the precipitate was filtrated, washed with n-heptane and collected to yield 76.2 gram suspended solid without the presence of “caked” solid paracetamol (50.2% yield) and with an average size of approximately 10μ. The induction time for crystals to be visually observed was less than 2 seconds after the addition started. S₁₀=4.5.

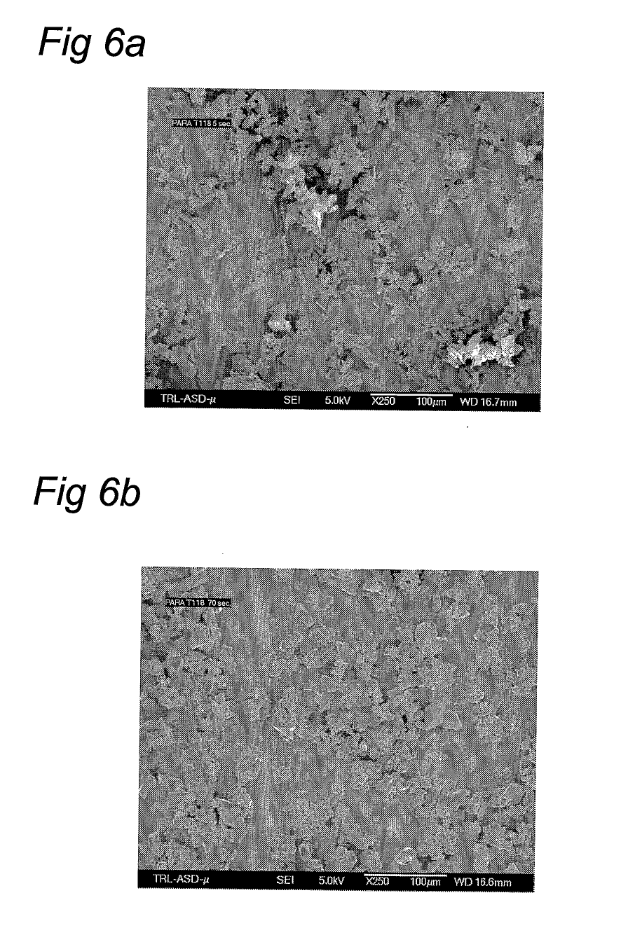

From FIG. 6 it is evident, that very small crystals were formed, the size of which did not change very much in the cause of the crystallization process. The method is not yet optimized for the yield.

### Inventive Example 2

Crystallization of pregnenolone from ethanol and water (reverse addition order);

using submerged feed into a square shaped mixing chamber with inventive agitation means.

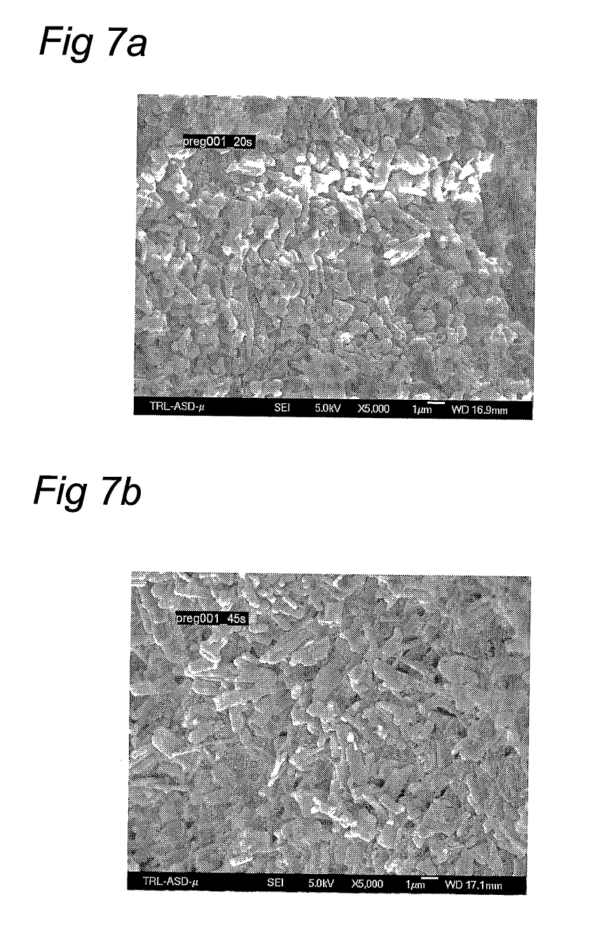

The vessel was filled with 2500 ml water. The initial temperature was controlled at 2° C. 42.5 Grams of pregnenolone dissolved in 1250 ml ethanol was added at a feed rate of 1000 ml/min equally divided over two inlets, controlled at 55° C. At the start of addition the mixing chamber and agitating device were completely immersed in the fluid present in the vessel. The mixing chamber and vessel are described in U.S. Pat. No. 4,289,733. In this example the stirring blade was as depicted in FIG. 9. Stirring was done at 15,000 rpm, creating tremendous turbulence in the mixing chamber. The impeller Reynolds number N_{Re} was 3.9*10⁶. The inlet position of both reactants was at opposite sides of the mixing chamber, below the agitating device. Simultaneous feeding of both reactant fluids was continued for 75 seconds, after which the precipitate was filtrated, washed with water and collected. FIG. 7 shows, that crystals with an average size of 1 to 2 μm were obtained without significant agglomeration and without caking of solid on the equipment surfaces. The induction time for crystals to be visually observed was less than 2 seconds after the addition started. S₁₀=200 (estimated).

### Inventive Example 3

Crystallization of pregnenolone from ethanol and water with medium rate addition (reverse addition order);

using submerged feed into a square shaped mixing chamber with inventive agitation means.

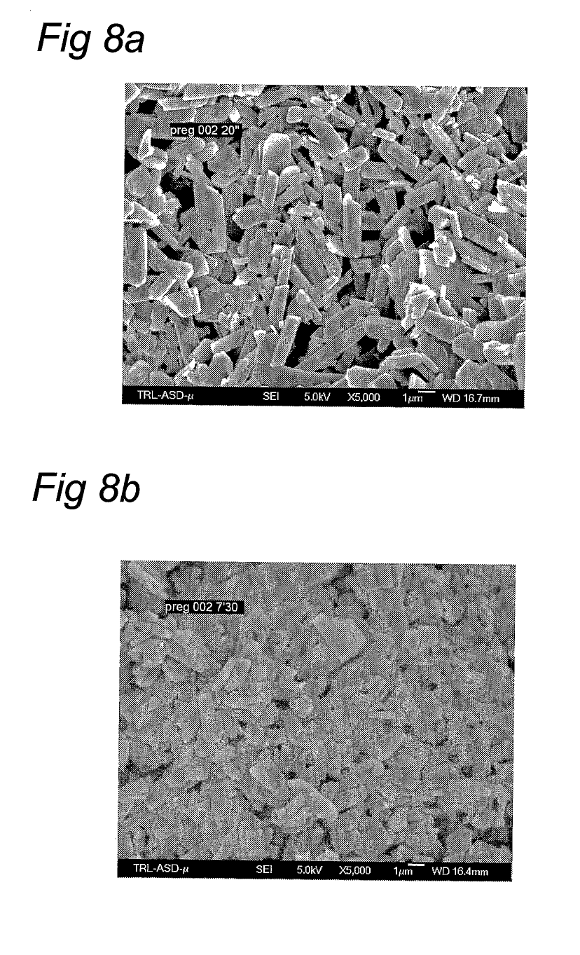

The vessel was filled with 1500 ml water. The initial temperature was controlled at 2° C. 25.5 Grams of pregnenolone dissolved in 750 ml ethanol was added at a feed rate of 100 ml/min equally divided over two inlets, thermostatted at 55° C. At the start of addition the mixing chamber and agitating device were completely immersed in the fluid present in the vessel. The mixing chamber and vessel are described in U.S. Pat. No. 4,289,733. The PTFE-coating was not applied in this case. In this example the stirring blade was as depicted in FIG. 9. Stirring was done at 15,000 rpm, creating tremendous turbulence in the mixing chamber. The impeller Reynolds number N_{Re} was 3.9*10⁶. The inlet position of both reactants was at opposite sides of the mixing chamber, below the agitating device. Simultaneous feeding of both reactant fluids was continued for 450 seconds, after which the precipitate was filtrated, washed with water and collected. Crystals with an average size of 1 to 10 μm were obtained without significant agglomeration and without caking of solid on the equipment surfaces, see FIG. 8. The induction time for crystals to be visually observed was less than 2 seconds after the addition started. S₁₀=80 (estimated).

Under identical conditions as applied in inventive examples 1, 2 and 3, the compounds progesterone, cortexolone, testosterone, hydrocortisone and desoxycorticosterone resulted in similar small crystals and narrow crystal size distributions and morphologies.

1-18. (canceled)

19. A method for the controlled precipitation of an organic compound comprising:
(a) obtaining a solution of said organic compound, and
(b) adding said solution via one or more inlets into a mixing chamber comprising one or more stirring means capable of providing isotropic mixing, the stirring means comprising one or more stirrer blades and a shaft,
wherein said mixing chamber is positioned (i) inside and in open connection with a vessel comprising a liquid in which the organic compound will not dissolve (non-solvent) and (ii) below the surface level of said non-solvent,
wherein the addition of said solution to the mixing chamber provides for an over-saturation S₁₀ of said organic compound in the mixture in said mixing chamber of more than 1.5 resulting in crystallisation and growth of crystals of said organic compound, and
wherein the stirring means provides for (i) isotropic mixing having a Reynolds number of at least 10⁶ and (ii) a residence time of said organic compound in said mixing chamber is longer than 0.1 milliseconds.

20. The method according to claim 1, wherein the residence time is between 0.1 milliseconds and 5 seconds,

21. The method according to claim 1, wherein the over-saturation S₁₀ is more than 5.

22. The method according to claim 1, wherein the over-saturation S₁₀ is more than 100.

23. The method according to claim 1, wherein the non-solvent is a mixture of solvents.

24. The method according to claim 1, further comprising the addition of a non-solvent for the organic compound to be precipitated in the mixing chamber at the same time and separately from the addition of the solution of the organic compound.

25. The method according to claim 1, wherein the organic compound to be precipitated is formed in the mixing chamber.

26. The method according to claim 1, wherein the stirrer blade and one or more inlets are positioned at the same height in the mixing chamber.

27. The method according to claim 1, wherein the stirrer blade and one or more inlets have a height difference not more than 30% of the height of the mixing chamber.

28. The method according to claim 1, wherein the diameter of the stirrer blade is at least 50% of the smallest dimension of the mixing chamber.

29. The method according to claim 1, wherein each second a volume of solution of the organic compound to be crystallised is added to the mixing chamber, which volume is more than 1% of the volume of the mixing chamber.

30. The method according to claim 1, wherein the mixing chamber and/or vessel is provided with a temperature control means.

31. The method according to claim 1, wherein the organic compound is a steroid hormone.

32. The method according to claim 13, wherein the steroid hormone is selected from the group consisting of Betamethasone, Betamethasone acetate, Betamethasone disodium phosphate, Chloroprednisone acetate, Corticosterone, Cortisone, Desoxycorticosterone, Desoxycorticosterone acetate, Desoxycorticosterone pivalate, Dexamethasone, Dichlorisone acetate, Fluocinolone acetonide, Fluorohydrocortisone, Fluorometholone, Fluprednisolone, Flurandrenolone, Hydrocortisone, Hydrocortisone acetate, Hydrocortisone sodium succinate, Methylprednisolone, Methylprednisolone sodium succinate, Paramethasone, Paramethasone acetate, Prednisolone, Prednisolone Phosphate sodium, Prednisolone pivalate, Prednisone, Triamcinolone, Triamcinolone acetonide, Triamcinolone diacetate, Androsterone, Fluoxymesterone, Methandrostenolone, Methylandrostenediol, Methyl testosterone, Norethandrolone, Oxandrolone, Oxymetholone, Prometholone, Testosterone, Testosterone cypionate, Testosterone enanthate, Testosterone phenylacetate, Testosterone propionate, Equilenin, Equilin, Estradiol, Estradiol benzoate, Estradiol cypionate, Estradiol dipropionate, Estriol, Estrone, Estrone benzoate, Ethynyl estradiol, Mestranol, Acetoxypregnenolone, Anagestone acetate, Chlormadinone acetate, Dimethisterone, Ethisterone, Ethynodiol diacetate, Flurogestone acetate, Hydroxymethylprogesterone, Hydroxymethylprogesterone acetate, Hydroxyprogesterone, Hydroxyprogesterone acetate, Hydroxyprogesterone caproate, Melengestrol acetate, Norethindrone, Norethindrone acetate, Norethisterone, Norethynodrel, Normethisterone, Pregnenolone, Progesterone, Aldosterone, Hydroxydione sodium, and Spironolactone.
